(19) United States
(12) Patent Application Publication
(10) Pub. No.: US 2021/0007967 A1
(43) Pub. Date: Jan. 14, 2021

(54) TOOTHPASTE

(71) Applicant: SHANGHAI JUKU COSMETIC CO., LTD., Shanghai (CN)

(72) Inventor: Qiaosheng LI, Shanghai (CN)

(21) Appl. No.: 16/504,571

(22) Filed: Jul. 8, 2019

(51) Int. Cl. A61K 8/9789; A61K 8/9794; A61Q 11/00

(52) U.S. Cl. CPC A61K 8/9789; A61Q 11/00; A61K 8/9794

(57) ABSTRACT

The present invention discloses a toothpaste, comprising a toothpaste body and an extract of Chinese medical composition which is included in a content of 2%-5%, the Chinese medical composition comprises by weight parts: Acorus calamus 20-30, ATRACTYLODES 22-30, Chrysanthemum 20-25 and Ginger 15-25.

### TECHNICAL FIELD

The present invention relates to a toothpaste, falling into the fields of Chinese medicine and daily necessity.

### BACKGROUND

Toothpaste is a cleaning product which is commonly used in daily life and has a long history. With the continuous development of science and technology and with the continuous improvement of process equipment, various types of toothpaste have come out successively, and the product quality and grade thereof have been continuously improved. Nowadays, the toothpaste has developed from a single cleaning-type toothpaste to a multi-functional toothpaste with a great variety of goods, functional diversity and hundreds of brands, to thereby meet the needs of different levels of consumption.

A medical toothpaste is prepared by adding some medicines into an ordinary toothpaste, and the medical toothpaste will reach the surface of the teeth or the environment around the teeth when the teeth are brushed, such that the dental plaque is reduced by the action of the medicine, thereby preventing tooth decay and periodontal disease. Medical toothpastes are very popular, and it is an ideal way to use toothpastes to generalize medicines with preventive or therapeutic effects.

Toothache is a common phenomenon in people's lives. Especially, toothache is an obviously-found disease after examination, and the toothache caused by poor diet is even more common. Toothache brings great inconvenience to people's lives.

However, there is a lack of an effective toothpaste capable of alleviating toothache in the prior art. Some medical toothpastes in the prior art can prevent dental decay, etc., but they are rarely involved in alleviating toothache symptoms.

Therefore, it is necessary to develop a toothpaste capable of alleviating toothache, which can effectively alleviate toothache by conventional tooth brushing.

### SUMMARY OF THE INVENTION

The object of the present invention is to provide toothpaste capable of effectively alleviating toothache. The toothpaste contains Chinese medical composition therein, so as to effectively alleviate toothache caused by various reasons just by tooth brushing.

The toothpaste according to the present invention comprises a toothpaste body and an extract of Chinese medical composition which is included in a content of 2%-5%, and the Chinese medical composition comprises by weight parts: Acorus calamus 20-30, ATRACTYLODES 22-30, Chrysanthemum 20-25 and Ginger 15-25.

The extract of Chinese medical composition is prepared by the following steps: pulverizing the Chinese medical composition medical materials into particle materials with a particle size of 2 mm; percolation extracting the particle materials by using 60% ethanol as a solvent at a percolation speed of 10 ml/min to obtain a percolation solution; vacuum evaporating the percolation solution to recover ethanol, concentrating the resultant to obtain a clear paste with a relative density of 1.1, and adjusting the pH value of the clear paste to be 8-9.5; and controlling to obtain an extract of Chinese medical composition with a yield of 70%-90%.

According to one embodiment of the present invention, wherein, the Chinese medical composition comprises by weight parts: Acorus calamus 30, ATRACTYLODES 30, Chrysanthemum 25 and Ginger 25.

According to one embodiment of the present invention, wherein, the Chinese medical composition comprises by weight parts: Acorus calamus 20, ATRACTYLODES 30, Chrysanthemum 25 and Ginger 20.

According to one embodiment of the present invention, wherein, the Chinese medical composition comprises by weight parts: Acorus calamus 25, ATRACTYLODES 23, Chrysanthemum 22 and Ginger 19.

A preparation method of above Chinese medical toothpaste comprises the following steps:

weighing, by weight parts, the individual Chinese medical raw materials, and mixing them to obtain a mixed Chinese medical composition medical materials; pulverizing the Chinese medical composition medical materials into particle materials with a particle size of 2 mm; percolation extracting the particle materials by using 60% ethanol as a solvent at a percolation speed of 10 ml/min to obtain a percolation solution; vacuum evaporating the percolation solution to recover ethanol, concentrating the resultant to obtain a clear paste with a relative density of 1.1, and adjusting the pH value of the clear paste to be 8-9.5; controlling to obtain an extract of Chinese medical composition with a yield of 70%-90%; and mixing above extract of Chinese medical composition with any conventional toothpaste body or any commercially available toothpaste body to obtain the toothpaste according to the present invention.

For use, it is only required to apply the toothpaste according to the present invention onto a toothbrush, and brush the teeth 2-3 times daily, to effectively alleviate toothache.

The toothpaste provided by the present invention can add the technical effect of relieving pain to the toothpastes in the prior art.

Other features and advantages of the present invention will be described in detail in the following embodiments.

### DETAILED DESCRIPTION OF THE EMBODIMENTS

The present invention provides numerous applicable inventive concepts that can be substantially embodied in the context. The specific examples described in the following embodiments of the present invention are merely used to exemplarily illustrate the specific implementations of the present invention and are not intended to limit the scope of the invention.

Without loss of generality, the present invention discloses a toothpaste, comprising a toothpaste body and an extract of Chinese medical composition which is included in a content of 2%-5%, wherein, the Chinese medical composition comprises by weight parts: Acorns calamus 20-30, ATRACTYLODES 22-30, Chrysanthemum 20-25 and Ginger 15-25.

All of the raw materials in the Chinese medical composition medical materials may be commercially available finished products, and also may be obtained by conventional preparation methods, for example, they may be prepared by drying and pulverizing raw materials into commonly-used powder in the art. The drying method may be baking or naturally drying, and preferably be naturally drying. The pulverizing method may be a commonly-used pulverizing method in the field of Chinese medicine, and the average particle size of the pulverized raw materials is preferably 20-30 mesh, and more preferably is 20 mesh.

In order to further implement above technical solution, the present invention provides a preparation method and an application method of above toothpaste as follows.

The above toothpaste is prepared by:

weighing, by weight parts, the individual Chinese medicine raw materials, and mixing them to obtain a mixed Chinese medical composition medical materials; pulverizing the Chinese medical composition medical materials into particle materials with a particle size of 2 mm; percolation extracting the particle materials by using 60% ethanol as a solvent at a percolation speed of 10 ml/min to obtain a percolation solution; vacuum evaporating the percolation solution to recover ethanol, concentrating the resultant to obtain a clear paste with a relative density of 1.1, and adjusting the pH value of the clear paste to be 8-9.5; controlling to obtain an extract of Chinese medical composition with a yield of 70%-90%; and mixing above extract of Chinese medical composition with any conventional toothpaste body or any commercially available toothpaste body.

For use, it is only required to apply the toothpaste according to the present invention onto a toothbrush, and brush the teeth 2-3 times daily, to effectively alleviate toothache.

Further, in one preferred technical solution of the present invention, the Chinese medical composition comprises by weight parts: Acorus calamus 30, ATRACTYLODES 30, Chrysanthemum 25 and Ginger 25.

Further, in one preferred technical solution of the present invention, the Chinese medical composition comprises by weight parts: Acorus calamus 20, ATRACTYLODES 30, Chrysanthemum 25 and Ginger 20.

Further, in one preferred technical solution of the present invention, the Chinese medical composition comprises by weight parts: Acorus calamus 25, ATRACTYLODES 23, Chrysanthemum 22 and Ginger 19.

Below, the present invention will be described in more detail by means of the examples.

### Examples 1-5

Examples 1-5 are used to illustrate the Chinese medical toothpaste provided by the present invention.

In the examples 1-5, the Chinese medical toothpastes are prepared according to the medical material ratios in Table 1.

TABLE 1

| | Acorus | | | |
|---|---|---|---|---|
| | calamus | ATRACTYLODES | Chrysanthemum | Ginger |
| Example | (g) | (g) | (g) | (g) |
| Example1 | 30 | 30 | 25 | 25 |
| Example2 | 20 | 30 | 25 | 20 |
| Example3 | 25 | 23 | 22 | 19 |
| Example4 | 23 | 26 | 23 | 15 |
| Example5 | 27 | 22 | 28 | 22 |

### Experimental Example

The experimental example is used for illustrating the technical effect of toothache-alleviating effect of the Chinese medical toothpaste provided by the present invention.

250 male or female experimenters (aged 20-60 years) were selected and randomly divided into 5 groups (with 50 experimenters in each group). The Chinese medical toothpastes 1-5 provided by the present invention were used for tooth brushing by the 5 groups 3 times daily (each for morning, afternoon and evening), respectively. After 1 week, the effects were observed. Compared with a condition before application, the experimenter whose toothache disappeared or was remarkably alleviated was marked as 0; the experimenter whose toothache was alleviated was marked as Δ; and the experimenter whose toothache had no remarkable alleviation was marked as x. The effects were shown in Table 2.

TABLE 2

| | Effects (number of experimenters) | | |
|---|---|---|---|
| Group | ∘ | Δ | x |
| 1 | 30 | 13 | 7 |
| 2 | 32 | 10 | 8 |
| 3 | 35 | 10 | 5 |
| 4 | 37 | 8 | 5 |
| 5 | 38 | 7 | 5 |

It can be seen from above experimental results that, the Chinese medical toothpaste provided by the present invention has a good effect in alleviating toothache.

1. A toothpaste, comprising a toothpaste body and an extract of Chinese medical composition which is included in a content of 2%-5%, wherein, the Chinese medical composition comprises by weight parts: Acorus calamus 20-30, ATRACTYLODES 22-30, Chrysanthemum 20-25 and Ginger 15-25.

2. The toothpaste according to claim 1, wherein, the Chinese medical composition comprises by weight parts: Acorus calamus 30, ATRACTYLODES 30, Chrysanthemum 25 and Ginger 25.

3. The toothpaste according to claim 1, wherein, the Chinese medical composition comprises by weight parts: Acorus calamus 20, ATRACTYLODES 30, Chrysanthemum 25 and Ginger 20.

4. The toothpaste according to claim 1, wherein, the Chinese medical composition comprises by weight parts: Acorus calamus 25, ATRACTYLODES 23, Chrysanthemum 22 and Ginger 19.
